(19) United States
(12) Patent Application Publication
(10) Pub. No.: US 2014/0194688 A1
(43) Pub. Date: Jul. 10, 2014

(54) CLOSED SUCTION SET INSERTABLE WITH BRONCHOSCOPE

(71) Applicant: PACIFIC HOSPITAL SUPPLY CO., LTD., Miaoli County (TW)

(72) Inventors: HSUAN-CHIAO HO, Miaoli County (TW); MING-CHUNG CHEN, Miaoli County (TW)

(73) Assignee: PACIFIC HOSPITAL SUPPLY CO., LTD., Miaoli County (TW)

(21) Appl. No.: 13/738,197

(22) Filed: Jan. 10, 2013

(51) Int. Cl. A61B 1/267; A61M 16/00; A61M 16/04; A61B 1/00; A61M 1/00

(52) U.S. Cl. CPC A61B 1/2676; A61B 1/00154; A61M 1/0023; A61M 16/0463; A61M 16/0057; A61M 1/0058
USPC 600/114

(57) ABSTRACT

A closed suction set insertable (1) includes a tube body (10), a bronchoscope connector (20), a choke valve (30) and a bronchoscope socket (40). The bronchoscope connector (20) is installed on the tube body (10), and the choke valve (30) is plugged into an external port (201) of the bronchoscope connector (20), and the choke valve (30) has a flexible choke plate (31) with an end extended into the bronchoscope connector (20) and sealed with an end of the choke valve (30). The bronchoscope socket (40) includes an external sleeve (41) and an internal sleeve (42), and the internal sleeve (42) has an insert hole (420) for passing a bronchoscope (2).

### FIELD OF THE INVENTION

The present invention relates to a closed suction device, and more particularly to the closed suction set insertable with a bronchoscope.

### BACKGROUND OF THE INVENTION

Bronchoscopy is a technique of visualizing the inside of airways for diagnostic and therapeutic purposes. A slender instrument (bronchoscope) is inserted into a patient's bronchus from the patient's nose or mouth, and images obtained from the bronchoscope are displayed on a screen to assist doctors to observe the condition of the patient's trachea. In addition, the bronchoscope is inserted from a cut opening of the trachea for patients who use a respirator or tracheostomy device.

Since the patients using a respirator or a tracheostomy device require a suction device for suction and oxygen delivery, therefore the suction device and its peripherals must be removed before inserting the bronchoscope, and then reinstall the suction device and peripherals after the bronchoscopy is completed. However, if a high-frequency bronchoscope is used, the processes of removing and reinstalling the suction device and peripherals repeatedly not only cause discomfort to patients, but also increase the workload of medical professionals.

In addition, a patient still needs an oxygen supply for bronchoscopy. When a bronchoscope is inserted into a present existing suction set, oxygen will leak from a gap between the bronchoscope and a tube body that may affect the normal supply of oxygen. Therefore, it is an important issue for the present invention to provide a convenient use of the suction device with the bronchoscope.

In view of the aforementioned problems, the inventor of the present invention based on years of experience in the related industry to conduct extensive researches and experiments, and finally provided a feasible design to overcome the aforementioned drawbacks of the prior art.

### SUMMARY OF THE INVENTION

Therefore, it is a primary objective of the present invention to provide a closed suction set insertable with a bronchoscope, and the suction set can be inserted with a bronchoscope to facilitate the operation of medical professionals, while reducing the patient's discomfort produced during the process of removing and installing the suction set.

Another objective of the present invention is to provide a closed suction set insertable with a bronchoscope, and the closed suction set can supply the required oxygen stably without leakage to a patient in a bronchoscopy.

To achieve the aforementioned objective, the present invention provides a closed suction set insertable with a bronchoscope, comprising a tube body, a bronchoscope connector, a choke valve and a bronchoscope socket. The tube body has a connected space, and the bronchoscope connector is disposed on tube body and interconnected to the connected space. The choke valve is plugged into an external port of the bronchoscope connector, and the choke valve has at least one flexible choke plate with an end extended into the bronchoscope connector and abutting one another, and sealed with an end of the choke valve. The bronchoscope socket includes an external sleeve sheathed on the bronchoscope connector and an internal sleeve formed and extended into the external sleeve, and the internal sleeve has an insert hole for passing and installing the bronchoscope.

Compared with the prior art, the closed suction set of the present invention has the bronchoscope connector installed on the tube body, the choke valve and the bronchoscope socket installed on bronchoscope connector. Since the bronchoscope socket comes with the insert hole for passing and installing the bronchoscope, therefore the bronchoscope can be inserted into the insert hole and entered into the tube body, and then passed out from the tube body and inserted into a human bronchus for the bronchoscopy. The present invention does not require removing the peripherals of the suction device for the bronchoscopy and thus can reduce the workload of installing the suction device and the bronchoscope by medical professionals. In addition, the closed suction set of the present invention has the choke valve for preventing possible leakage of oxygen, so that the present invention can supply oxygen to patients normally during the bronchoscopy to improve the practicality of the invention.

### BRIEF DESCRIPTION OF THE DRAWINGS

FIG. 1 is an exploded view of a closed suction set of the present invention;

FIG. 2 is a schematic view of a partial assembly at a bronchoscope connector of the present invention;

FIG. 3 is a cross-sectional view of a closed suction set of the present invention;

and

FIG. 4 is a schematic view, showing an application of a closed suction set inserted with a bronchoscope in accordance with the present invention.

### DESCRIPTION OF THE PREFERRED EMBODIMENTS

The technical contents of the present invention will become apparent with the detailed description of preferred embodiments accompanied with the illustration of related drawings as follows.

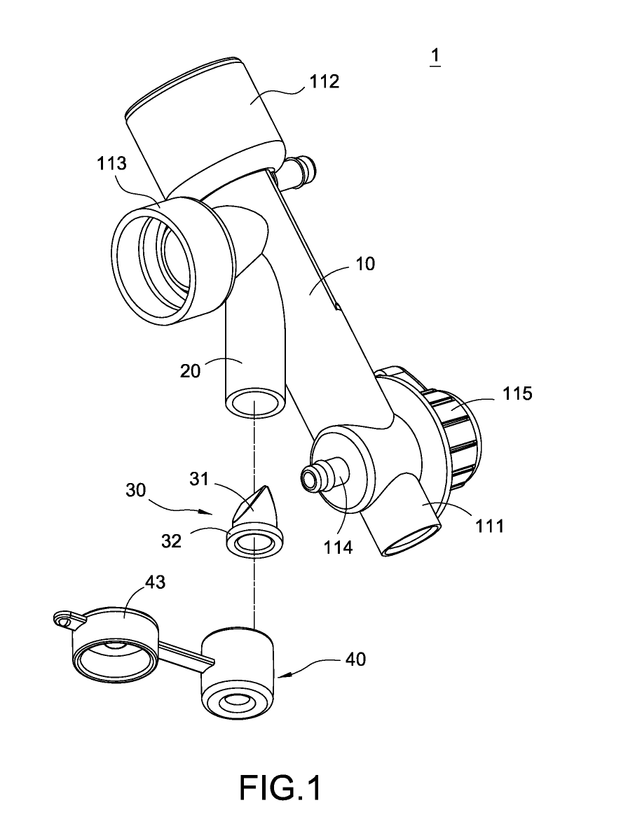

With reference to FIGS. 1 to 3 for an exploded view, a schematic view and a cross-sectional view of a closed suction set insertable with a bronchoscope in accordance with the present invention respectively, the closed suction set 1 comprises a tube body 10, a bronchoscope connector 20, a choke valve 30 and a bronchoscope socket 40.

The tube body 10 has a connected space 100 and a plurality of connectors interconnected with one another through the connected space 100. The connectors include a suction connector 111 for connecting a suction tube, a tracheal connector 112 for connecting a human trachea, a respiration connector 113 for connecting an oxygen supply device, and a flush connector 114 and a control valve connector 115 installed between the respiration connector 113 and the suction connector 111, and the flush connector 114 is connected to a flush device. It is noteworthy that the aforementioned connectors are prior arts, and thus will not be described here.

The bronchoscope connector 20 is installed on the tube body 10 and interconnected to the connected space 100, and the bronchoscope connector 20 is installed with an inclined angle with respect to the tube body 10. In this preferred embodiment, the bronchoscope connector 20 is situated between the respiration connector 113 and the flush connector 114, but the invention is not limited to such arrangement only.

The choke valve 30 is plugged onto an external port 201 of the bronchoscope connector 20. The choke valve 30 has at least one flexible choke plate 31 with an end extended into the bronchoscope connector 20 and abutting with each other. In this preferred embodiment, the quantity of the flexible choke plates 31 is plural, and the flexible choke plates 31 are enclosed to form a conical shape and sealed with an end of the choke valve 30. In a preferred embodiment of the present invention, the choke valve 30 includes a ring 32 sheathed onto an external port 201 of the bronchoscope connector 20, and the other end of the flexible choke plate 31 is extended and coupled to the ring 32.

In addition, the bronchoscope socket 40 includes an external sleeve 41 sheathed on the bronchoscope connector 20 and an internal sleeve 42 formed and extended in the external sleeve 41, and the internal sleeve 42 has an insert hole 420. In addition, the bronchoscope socket 40 further includes a cover 43 covered onto the external sleeve 41 and coupled onto an external wall of the external sleeve 41. Preferably, the cover 43, the external sleeve 41 and the internal sleeve 42 are integrally formed by injection molding.

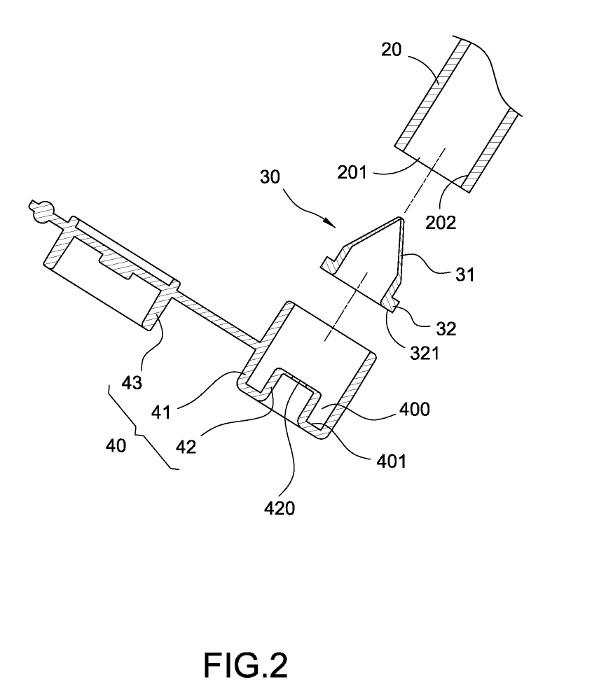

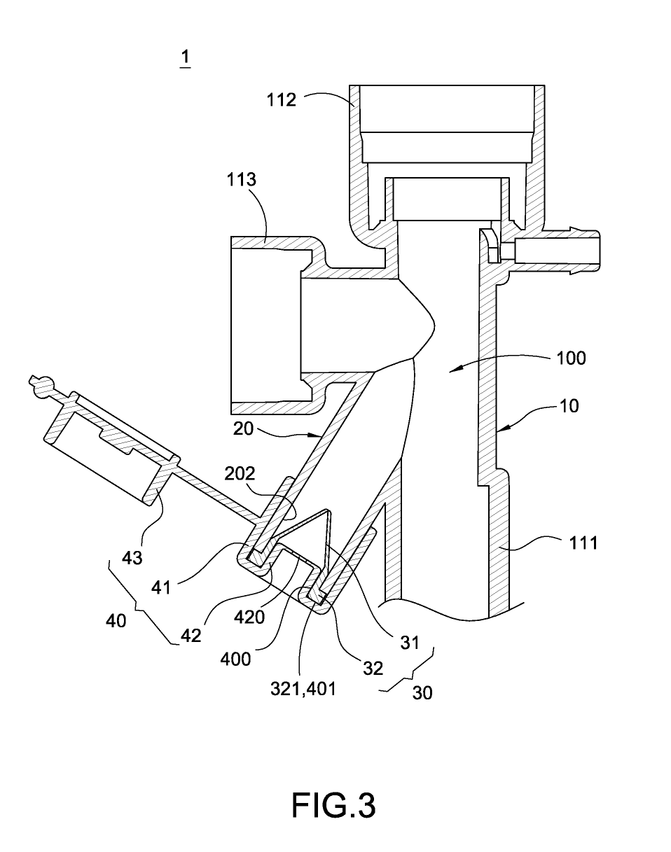

In a preferred embodiment as shown in FIGS. 2 and 3, a circular containing groove 400 is formed between the external sleeve 41 and the internal sleeve 42 of the bronchoscope socket 40, and the ring 32 is installed in the circular containing groove 400, and a sidewall of the ring 32 is attached on an internal wall 202 of the bronchoscope connector 20 and an internal sleeve 42 of the bronchoscope socket 40. More specifically, the ring 32 is clamped between the bronchoscope connector 20 and the bronchoscope socket 40, and a top surface 321 of the ring 32 is clamped between the external port 201 of the bronchoscope connector 20 and an internal wall 401 of the bronchoscope socket 40.

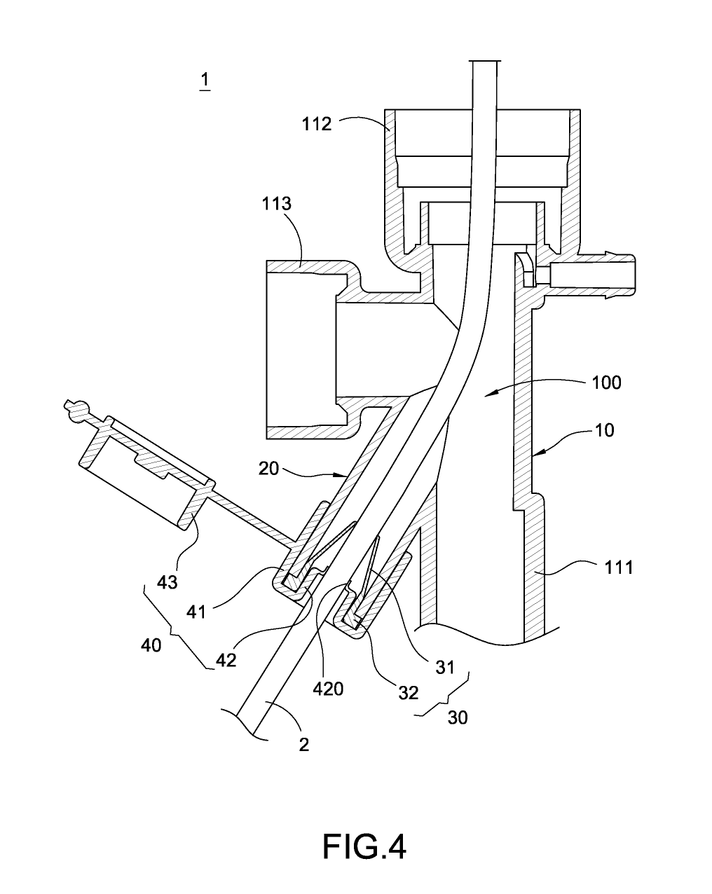

With reference to FIG. 4 for a schematic view, showing an application of a closed suction set inserted with a bronchoscope in accordance with the present invention, the closed suction set 1 is capable of receiving an insertion of a bronchoscope 2. The internal sleeve 42 of the bronchoscope socket 40 has an insert hole 420 for passing and installing the bronchoscope 2, and the bronchoscope 2 is inserted into the closed suction set 1 from the insert hole 420, and then extended into the choke valve 30, and passed out from the flexible choke plates 31 and entered into the connected space 100, and finally passed out from the tracheal connector 112, so as to be inserted into a human bronchus for a checkup.

It is noteworthy that the insert hole 420 has a diameter smaller than the diameter of the bronchoscope 2 in a preferred embodiment of the present invention, so that the internal wall of the internal sleeve 42 is closely attached to the bronchoscope 2, and the flexible choke plates 31 are closely attached to the external wall of the bronchoscope 2 as shown in FIG. 4. With such arrangement, the bronchoscope 2 can be inserted into a human trachea through the closed suction set 1, while preventing oxygen in the closed suction set 1 from leaking, so as to supply the required oxygen to patient normally during a bronchoscopy.

While the invention has been described by means of specific embodiments, numerous modifications and variations could be made thereto by those skilled in the art without departing from the scope and spirit of the invention set forth in the claims.

What is claimed is:

1. A closed suction set insertable with a bronchoscope (1), comprising:
a tube body (10), having a connected space (100);
a bronchoscope connector (20), installed on the tube body (10) and interconnected with the connected space (100);
a choke valve (30), plugged into an external port (201) of the bronchoscope connector (20), and having at least one flexible choke plate (31) with an end extended into the bronchoscope connector (20) and abutted against the bronchoscope connector (20) and sealed with an end of the choke valve (30); and
a bronchoscope socket (40), including an external sleeve (41) sheathed on the bronchoscope connector (20) and an internal sleeve (42) formed and extended into the external sleeve (41), and the internal sleeve (42) having an insert hole (420) for passing the bronchoscope (2).

2. The closed suction set insertable with a bronchoscope according to claim 1, wherein the tube body (10) includes at least one connector communicated with one another through the connected space (100), and the connectors include a suction connector (111) for connecting a suction tube, a tracheal connector (112) adapted to be for connecting a human trachea, a respiration connector (113) for connecting an oxygen supply device, a flush connector (114) installed between the respiration connector (113) and the suction connector (111), and a control valve connector (115), and the bronchoscope connector (20) is situated between the respiration connector (113) and the flush connector (114).

3. The closed suction set insertable with a bronchoscope according to claim 1, wherein the bronchoscope connector (20) is installed with an inclined angle with respect to the tube body (10).

4. The closed suction set insertable with a bronchoscope according to claim 1, wherein the choke valve (30) includes a ring (32) sheathed and pressed on external port (201) of the bronchoscope connector (20), and the other end of the flexible choke plate (31) is extended and coupled to the ring (32).

5. The closed suction set insertable with a bronchoscope according to claim 4, wherein the ring (32) is clamped between the bronchoscope connector (20) and the bronchoscope socket (40).

6. The closed suction set insertable with a bronchoscope according to claim 5, wherein the ring (32) has a top surface clamped between the external port (201) of the bronchoscope connector (20) and the internal wall (401) of the bronchoscope socket (40).

7. The closed suction set insertable with a bronchoscope according to claim 5, wherein the ring (32) has a sidewall attached onto the internal wall (202) of the bronchoscope connector (20) and the internal sleeve (42) of the bronchoscope socket (40).

8. The closed suction set insertable with a bronchoscope according to claim 4, further comprising a circular containing groove (400) formed between the external sleeve (41) and the internal sleeve (42) of the bronchoscope socket (40), and the ring (32) being installed in the circular containing groove (400).

9. The closed suction set insertable with a bronchoscope according to claim 1, wherein the flexible choke plate (31) comes with a plural quantity, and the flexible choke plates (31) are enclosed to form a conical shape.

10. The closed suction set insertable with a bronchoscope according to claim 1, wherein the insert hole (420) has a diameter smaller than the diameter of the bronchoscope (2).

11. The closed suction set insertable with a bronchoscope according to claim 1, wherein the bronchoscope socket (40) further includes a cover (43) covered onto the external sleeve (41) and coupled to an external wall of the external sleeve (41).
